(19) United States
(12) Patent Application Publication
(10) Pub. No.: US 2003/0069174 A1
(43) Pub. Date: Apr. 10, 2003

(54) Use of human alpha1-acid glycoprotein for producing a pharmaceutical preparation

(76) Inventors: Ludwig Pichler, Vienna (AT); Eva-Maria Muchitsch, Vienna (AT); Anton Philapitsch, Ebenfurt (AT); Hans-Peter Schwarz, Vienna (AT); Yendra Linnau, Vienna (AT); Wolfgang Teschner, Vienna (AT)

Correspondence Address: HELLER EHRMAN WHITE & MCAULIFFE LLP, 1666 K STREET,NW, SUITE 300, WASHINGTON, DC, 20006, US

(21) Appl. No.: 10/298,942

(22) Filed: Nov. 19, 2002

Related U.S. Application Data
(63) Continuation of application No. 09/380,921, filed on Nov. 23, 1999.
Application No. 09/380,921 is a 371 of international application No. PCT/AT98/00060, filed on Mar. 10, 1998.

(30) Foreign Application Priority Data
Mar. 10, 1997 (AT) A 409/97

(51) Int. Cl. A61K 38/17

(52) U.S. Cl. 514/8

(57) ABSTRACT

The invention relates to the use of human α₁-acid glycoprotein (AGP) for producing a pharmaceutical preparation for treating non-inflammatory disturbances of circulation or microcirculation, respectively.

The invention relates to new medical uses of orosomucoid.

α₁-Acid glycoprotein (AGP), also called orosomucoid, is a substance recovered from plasma, having a molecular weight of 40,000 Daltons, and comprising a carbohydrate portion of between 30 and 50%. AGP consists of a single polypeptide chain of 183 amino acids and comprises two disulfide bonds. Furthermore, it comprises five carbohydrate chains all located in the first half of the peptide chain. These carbohydrate groups consist of about 14% of neutral hexoses, 14% of hexosamines, 11% of sialic acid and 1% of fructose. Depending on the source of the AGP preparation and on the recovery or characterisation methods used, AGP appears in different forms which is attributed to differences in the polypeptide chain as well as to differences in the carbohydrate chain.

The properties and biological functions of orosomucoid have been described in the survey articles by Schmid (in “The Plasma Proteins Structure Function and Genetic Control”, Vol. 1 (1975), Academic Press, Ed. Frank A. Putnam, 2nd Edition, pp. 183-228) and Kremer et al. (Pharmacological Reviews 40 (1988), pp. 1-47).

In the medical field, AGP so far has been considered as an essential carrier substance for predominantly basic medicaments in plasma (cf. Kremer et al.).

Furthermore, orosomucoid could be demonstrated to have a positive effect on inflammatory reactions. Thus, Denko et al. (Agents and Actions 15, 5/6 (1984), 539-540) have described an antiinflammatory effect of AGP in urate crystal inflammations in rats. Libert et al. (J. Exp. Med. 180 (1994), 1571-1575) proved that a similar indication lies in preventing septic shock in connection with the effect of TNF-α or lipopolysaccharides.

To improve perfusion disturbances, in particular of microcirculation, as well as reperfusion damages so far either vasoactive substances or special blood factors which influence hemostatis or fibrinolysis, respectively, in particular anticoagulants or thrombolytically active factors have been administered, or a corresponding volume substitution has been carried out.

To treat hypovolemic shock conditions occurring independently of inflammatory reactions, volume substitution has been effected so far, whereby albumin solutions commonly having been used.

It is the object of the present invention to provide new medical indications for orosomucoid.

Surprisingly it has been found that orosomucoid is suitable for the treatment of disturbed circulation, or microcirculation, respectively, of the non-inflammatory type. Thus, it can be used to improve perfusion disturbances, in particular a disturbed microcirculation, as well as reperfusion injuries, and, above all, in shock conditions, for a better supply of the vital organs, such as brain, lung, heart, liver and kidney.

These indications are all of the non-inflammatory type, i.e. the disturbances are indicated if they do not occur in connection with SIRS (“systemic inflammatory response syndrome”). For a definition of SIRS, cf. Critical Care Medicine 20 (6), 864-874 (1992). Due to inflammations, cells and tissue are directly damaged, and as a consequence the permeability of the vessels and the circulation are disturbed. With the inventive use of AGP for producing a pharmaceutical preparation for the treatment of disturbances of circulation, and microcirculation, respectively, however, these disturbances are of the non-inflammatory Lype and thus have different causes. Among these causes are altered pressure conditions, particularly in connection with a reduction of intravasal volume. If not treated, the circulatory disorders triggered thereby will lead to hypovolemic shock. Triggering mechanisms in this case are considered to be acute hemorrhages, excessive loss of liquid, such as by vomiting, diarrhoea, extreme sweating, dehydration, excessive discharge of urine, peritonitis, pancreatitis, ischemias in the splanchnic region, ileus, gangrene, blunt traumas, damage of large groups of muscles, or burns.

So far, these conditions commonly have been treated with dextrane solutions, hydroxy ethyl starch, Ringer's lactate or albumin solutions. However, these substances do not possess any antiinflammatory properties, and thus it has been surprising that orosomucoid which had been used for the treatment of inflammations could assume this function in the indications according to the invention.

According to the invention, orosomucoid can also be used in case of a relative hypovolemia. The latter is found if the absolute blood volume is not reduced, yet there exists an undersupply of the organs. The reason for this may reside in a vasodilatory change which may be neurogenic, metabolic, toxic or humoral. A further reason is an increased vessel permeability, possibly of the anaphylactic type or caused by diverse snake venoms.

Likewise, a pump failure may be the cause of hypovolemic shock, caused by an acute myocardial infarction, myocarditis, or a highly reduced output performance, acute valvular incompetence, myocardial rupture, septum perforation, arrhythmias, such as bradycardia, tachycardia or fibrillation, or a mechanical compression of the heart, respectively, or physical obstacles, e.g. thrombi or embolisms.

The present invention therefore also relates to the use of AGP for producing a preparation for the treatment of hemorrhagic and/or hypovolemic shock and for stabilizing the intravasal volume, in particular in case of acute hemorrhages, excessive liquid loss, or vasodilation, respectively

According to the invention, AGP can also be used to prevent reperfusion damages as a consequence of a stroke, in particular for reducing cerebral oedema. Reperfusion injuries occur primarily after removal of a flow obstacle, e.g. occlusion of a vessel due to deposits or blood clots. Such injuries are particularly found as a consequence of a stroke, where a cerebral oedema is formed, leading to neurological dysfunctions. Tissue damage in transplanted organs possibly occurring due to re-started perfusion are also among the reperfusion injuries.

A further disturbance of microcirculation which, according to the invention, can be treated by AGP, are the microcirculation disturbances in vital organs, in particular in the kidney, which may, e.g., directly cause proteinuria.

Microcirculation disturbances in organs may, however, also be caused by oedemas. Therefore, the present invention also relates to the use of orosomucoid for producing a preparation for the prevention or treatment, respectively, of oedemas.

The mode of production of AGP is known (e.g. from WO 95/07703). As the source of human AGP, preferably human plasma or a plamsma fraction, respectively, e.g. a COHN fraction, such as COHN IV or COHN V, will be used. According to the invention, the preparation advantageously is produced as a storage-stable infusion solution, and preferably it is provided as a lyophilisate.

The dose used will depend on the respective indication and also on the severity of the patient's condition, e.g. on the amount of blood lost. As a rule, a single dose in the range of from 70 mg/kg body weight to 5 g/kg will be used, the range from 100 to 700 mg/kg being particularly preferred.

The application according to the invention may be of any type, preferred are i.v., s.c., i.m. and a local application.

Advantageously, the pharmaceutical preparation used contains at least 50% orosomucoid, preferably more than 70%, in particular more than 90%, based on the total protein. As a further component, the pharmaceutical preparation according to the invention may furthermore contain albumin and A₁AT (α₁-antitrypsin).

Preferably a stabilizer, in particular sodium caprylate and, optionally, tensides are admixed to the pharmaceutical preparation before its use according to the invention, so as to increase its storage stability and its stability during a heat treatment, respectively.

It is also suitable to treat the pharmaceutical preparation for an inactivation or depletion of viruses, respectively, in particular by at least one physical treatment, such as a heat treatment and/or filtration. To inactivate viruses, a number of physical, chemical or chemical-physical methods are known, such as, e.g., a heat treatment, e.g. according to EP 0 159 311 A or EP 0 637 451 A, a hydrolase treatment according to EP 0 247 998 A, or a radiation treatment or a treatment with an organic solvent and/or tensides, e.g. according to EP 0 131 740 A. Further suitable virus inactivation steps in the production of the preparations according to the invention are described in EP 0 506 651 A or in WO 94/13329 A.

For certain indications, i.e. in particular hypovolemic or neurogenic shock, the administration of orosomucoid advantageously is combined with the administration of vasoactive substances (constringent or dilating), which may be administered either together or in parallel.

Therefore, according to a further aspect, the present invention relates to an infusion preparation for the treatment of shock conditions, containing AGP and a vasoactive substance as active components.

According to the invention, this infusion preparation is provided in the form of a kit which comprises

AGP in a pharmaceutical preparation, and

a vasoactive substance, optionally in separate containers.

Application of AGP according to the invention may be prophylactic, yet, primarily, therapeutic.

The invention will now be explained in more detail by way of the following Examples and the drawing figures, to which, however, it shall not be restricted.

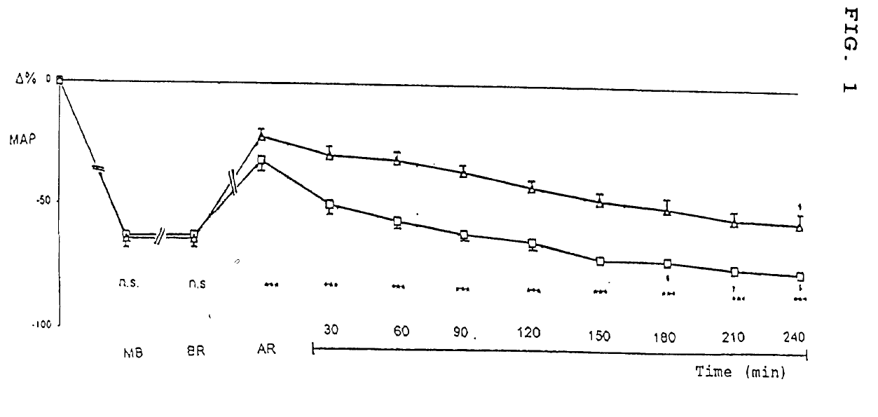

FIGS. 1 to 5 show the results of the treatment of hemorrhagic shock in the rat model;

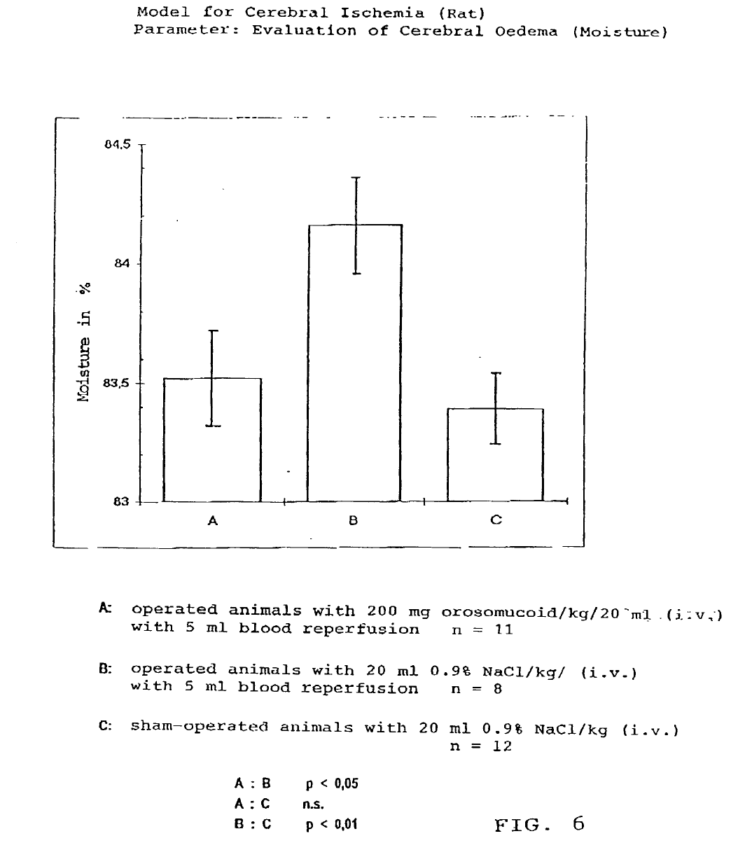

FIGS. 6 to 8 show the results in the prevention of cerebral oedema in the stroke model on the rat; and

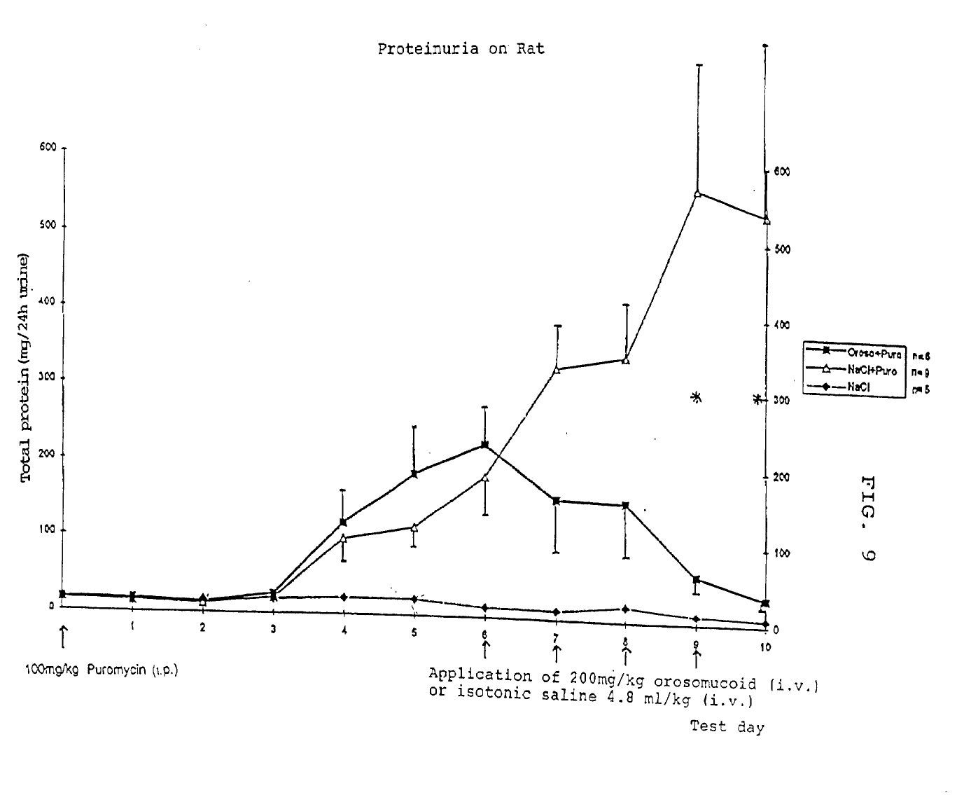

FIG. 9 shows the results of the treatment of proteinuria in a rat model.

### EXAMPLE 1

### Treatment of Hemorrhagic Shock in the Rat Model (at Present Considered by Applicant to Be the Best Mode of Carrying Out the Invention)

These experiments have been carried out in a manner analogous to Wang and Chaudry (J. Surg. Res 50 (1991), 163-169). The animals were fasted over night, yet had free access to water. As an introduction to anesthesia, the animals were injected i.m. with 60 mg/kg pentobarbital. Anesthesia was maintained by 5 mg of pentobarbital per animal every 1.5 h, s.c. The trachea was cannulated for artificial respiration in emergencies. A polyethylen catheter was provided in the left jugular vein for infusions (volume substitution) and injections. A second catheter was introduced via the right jugular vein into the right atrium to inject cold isotonic saline (≦20° C.; thermodilution method) Via the right carotid, a thermocouple was advanced into the aorta arch to take the blood temperature. Both femoral arteries were cannulated, one for determining the blood pressure, the other one for withdrawing blood. During the entire experiment, the body temperature was maintained at 36.5° by using a rectal thermometer which was connected to an infrared lamp. To set a trauma prior to bleeding, after depilation, a 5 cm laparotomy was carried out in the linea alba by means of an electrocauteriser. This cut subsequently was closed in layers.

1 IU of heparin/g body weight was injected. Subsequently, the mean arterial blood pressure was lowered to 40 mmHg within 10 min by withdrawing blood from the femural artery. The blood pressure was maintained for a maximum of 80 min at 40 mmHg either by further withdrawing of blood or by injection of Ringer's solution in a total volume not exceeding 40% of the lost blood. After these 80 min or earlier (if the blood pressure could no longer be kept above 40 mmHg), volume substitution was started by replacing the 3-fold volume of the total blood loss with Ringer's solution during 60 min. Volume substitution was followed by an observation period of 4 h. The surviving animals were sacrificed with an overdose of pentobarbital, i.v.

The mean arterial blood pressure was continuously registered by means of a polygraph by using an electromechanical pressure transducer. The heart rate was continuously recorded by pulse waves. The cardiac output per minute was determined by means of the thermodilution method, 200 μl of cold saline being injected into the right atrium. By taking the blood temperature in the aorta arch, the thermodilution curve was integrated by means of a Cardiomax II (Model 85, Columbus Instruments), and the cardiac output per minute was given in ml/min. The systolic volume and the entire peripheral vascular resistance were calculated by dividing the cardiac output per minute by the heart rate, or by dividing the blood pressure by the cardiac output per minute, this ratio being multiplied by 10³ (mmHg.ml.min⁻¹.10³) Initial values were indicated as the natural values. All other values were given in % of the respective initial value (Δ%). Mean values±standard error were also calculated. The significance of the differences between initial values and all other values was verified by the t-test for paired observations. For a comparison between the groups, the “double-side t-test” was employed.

It has been shown that in all animals (n=30), the amount of blood withdrawn was 7.0±2.9 ml, to reduce the mean arterial blood pressure to 40 mmHg. The blood pressure could be kept at this low level for 77.8±1.5 min. The blood pressure drop was accompanied by a lowering of the cardiac output per minute, systolic volume, and the entire peripheral vascular resistance. The heart rate dropped in all three groups, with an initial rise in the AGP-treated group.

Volume substitution in control animals (n=13) with Ringer's solution i.v. (volume: 3 times the amount of lost blood) could not re-establish the mean arterial blood pressure which had dropped significantly over the entire observation period (range −50.5±2.3% to −63.6±10.3%). A slight drop in the heart rate could also be observed which became significant 180 to 240 min after volume substitution (maximum: −29.9±8.8% at 240 min). The cardiac output per minute could be returned to the initial values immediately upon volume substitution, yet between 30 and 240 min thereafter it was significantly lowered (range −25.9±5.5% to −51.4±7.4%). The same course over time could be observed for the systolic volume (range of drop: −26.7±8.8% to −31.1±9.9). The entire peripheral vascular resistance dropped during the observation period (range 19.1±18.2% to 39.7±9.9%), the differences being significant 30 to 120 min after reanimation. Three animals died≦150 min after substitution of the volume and were not included in the evaluation. Further three animals died after 180 min.

Two further groups were treated by using AGP or a placebo formulation, respectively, instead of Ringer's solution. The AGP solution (200 mg/kg), purified from COHN fraction V from human plasma by precipitation and further pasteurization at 60° C. for 10 h, and the analogous amount of placebo formulation (albumin solution from human albumin, IMMUNO, by separating orosomucoid) were diluted with Ringer's solution to the three-fold amount of the individual blood loss.

AGP was tested on 14 animals; 1 rat died during treatment (volume substitution), 3 rats died within less than 150 min after treatment, and 1 animal died 180 min after volume substitution. The placebo formulation was administered to 18 animals. Four of these animals died during treatment, four died within less than 150 min after treatment, four animals died after more than 180 min after volume substitution. Rats which died within less than 150 min after treatment were not included in the evaluation.

A comparison of the results obtained by Ringer's solution with those obtained by the placebo formulation showed that equal or lower values of the mean arterial blood pressure, heart rate and entire peripheral vascular resistance were obtained with the placebo formulation treatment. The values for cardiac output per minute and systolic volume were equal or higher in the placebo formulation group than in the Ringer's solution group.

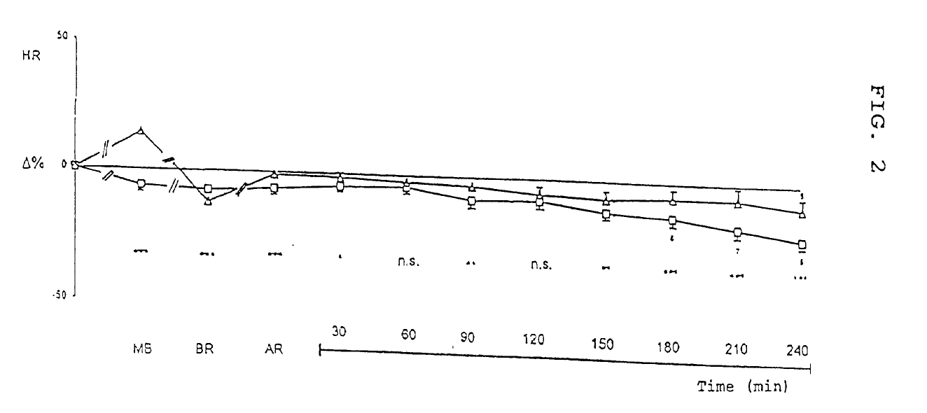

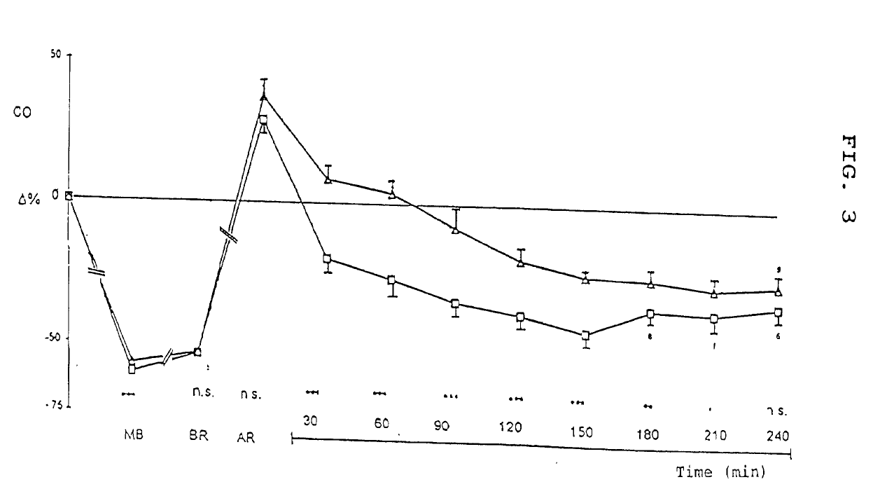

In animals which had been treated with AGP, the blood pressure rose initially and then dropped gradually (cf. FIG. 1). Complete restoration of the blood pressure, however, could not be achieved. All the values in the observation period were lower than the initial values (p≦0.001). As regards the heart rate (FIG. 2), no change in the period after volume substitution as compared to the initial values could be observed (p>0.05). The cardiac output per minute (FIG. 3) was higher than the initial values immediately after volume substitution (p<0.01), yet re-adjusted to the initial values (+30 to +90 min; p>0.05), and finally dropped to below the initial values (p<0.05 or ≦0.01).

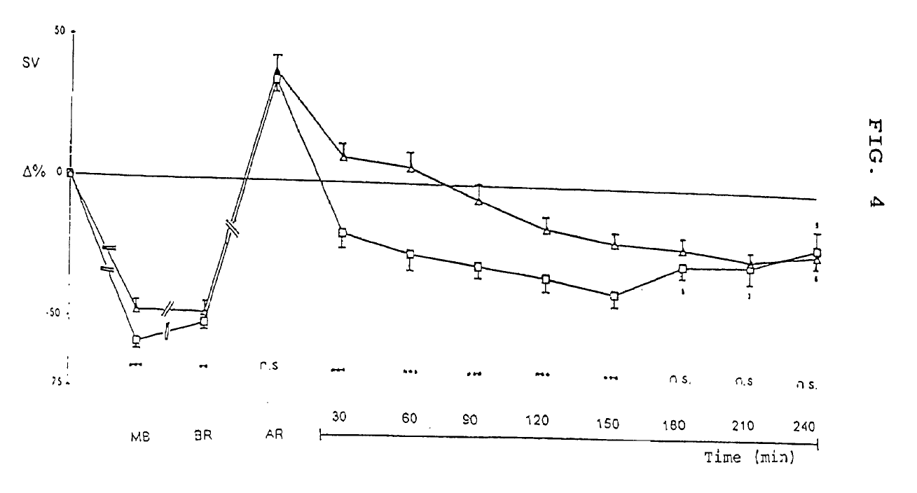

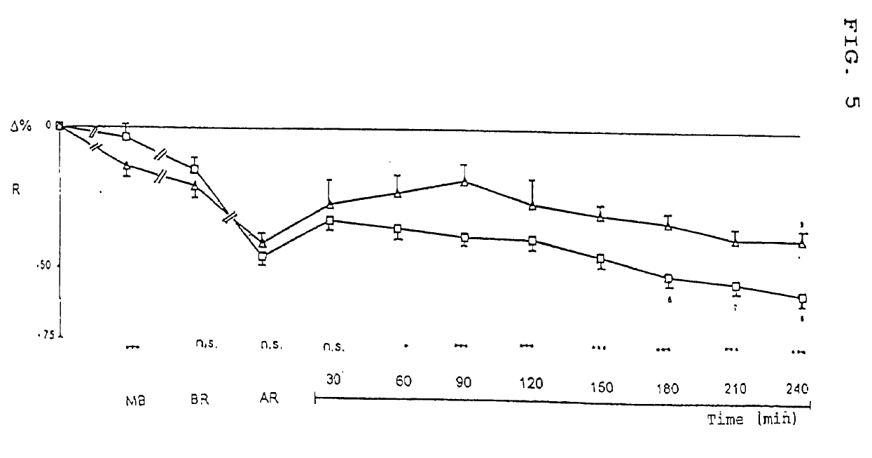

For the systolic value, the situation was similar (FIG. 4), except that the values during the first 120 min were statistically not different from the initial values. The entire peripheral vascular resistance (FIG. 5) was lower over the entire observation period as compared to the initial values; however, a significance was not reached at +30, +90 and +120 min.

FIGS. 1 to 5 show also the comparison between animals which had been treated with AGP and placebo formulation. The mean arterial blood pressure is significantly higher at all points of measurement after volume substitution in the AGP-treated group (FIG. 1). The heart rate is equal or significantly higher in the AGP-group. Differences, however, are very small (FIG. 2). The cardiac output per minute is significantly higher in the AGP-treated group, except for the point of measurement “240 min” after volume substitution (FIG. 3). The systolic volume is significantly higher after treatment with AGP at 30 to 150 min after volume substitution (FIG. 4). The entire peripheral vascular resistance is increased in the AGP-group—as compared to the placebo formulation—at 60 to 120 min after infusion (FIG. 5).

These experiments demonstrate the superiority of a treatment with AGP as compared to a placebo formulation (containing the same protein amount in the form of albumin which is tree from AGP) or Ringer's solution. Hence it follows that AGP can maintain the perfusion of vital organs in case of hypovolemic shock.

### EXAMPLE 2

### Prevention of Cerebral Oedema in the Stroke Model on Rat

A global cerebral ischemia (“stroke”) was caused by clamping both carotids and withdrawing 5 ml of blood. After 30 min of ischemia, the carotids were re-opened, and the withdrawn blood was re-infused. 23.5 h later the animals were sacrificed and the water content of the two halves of the cerebrum was determined.

In previous experiments it had been found that orosomucoid at 600 mg/kg i.v. is capable of preventing the formation of cerebral oedema following global cerebral ischemia. The formulation buffer had remained without such effect. In the present example, a dose response and time effect relationship is set up for the oedema-preventing effect of orosomucoid.

Orosomucoid which had also been used in example 1 was tested on rats at 200 mg/kg i.v., with simultaneous blood reperfusion The results appear from FIG. 6. It has been shown that pseudo-operated animals (C, n=12) do not have a cerebral oedema, while ischemic, saline-treated animals exhibit massive cerebral oedema (B, n=8). Ischemic animals which had been treated with orosomucoid (A, n=11) again behaved like the sham-operated animals.

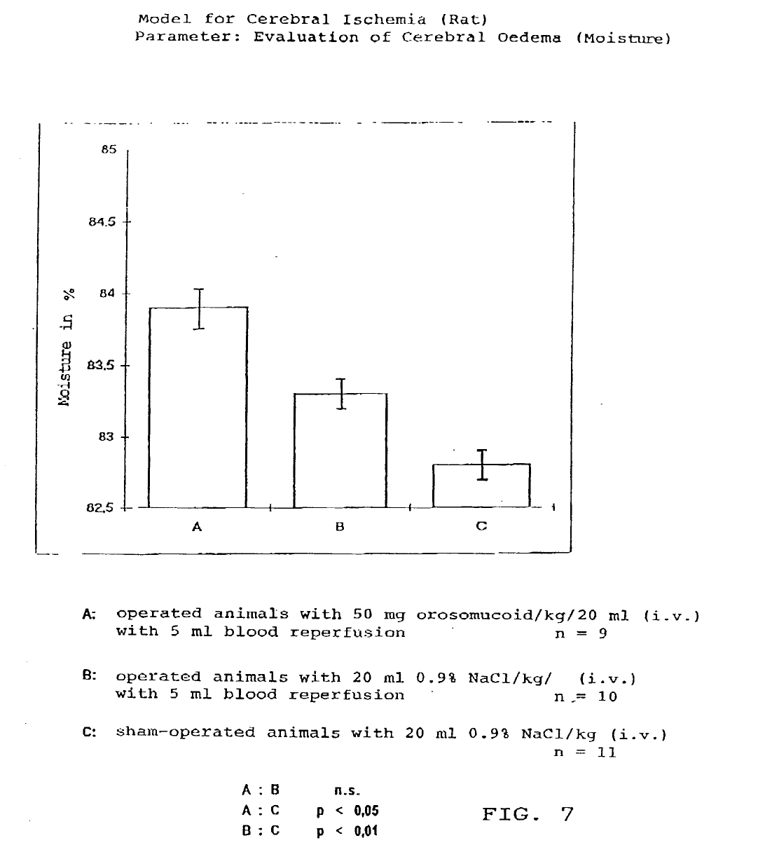

However, when the dose was reduced to 50 mg of orosomucoid per kg, i.v., a protective effect could no longer be found (cf. column A in FIG. 7), whereby the dose dependence of the effect has been proven.

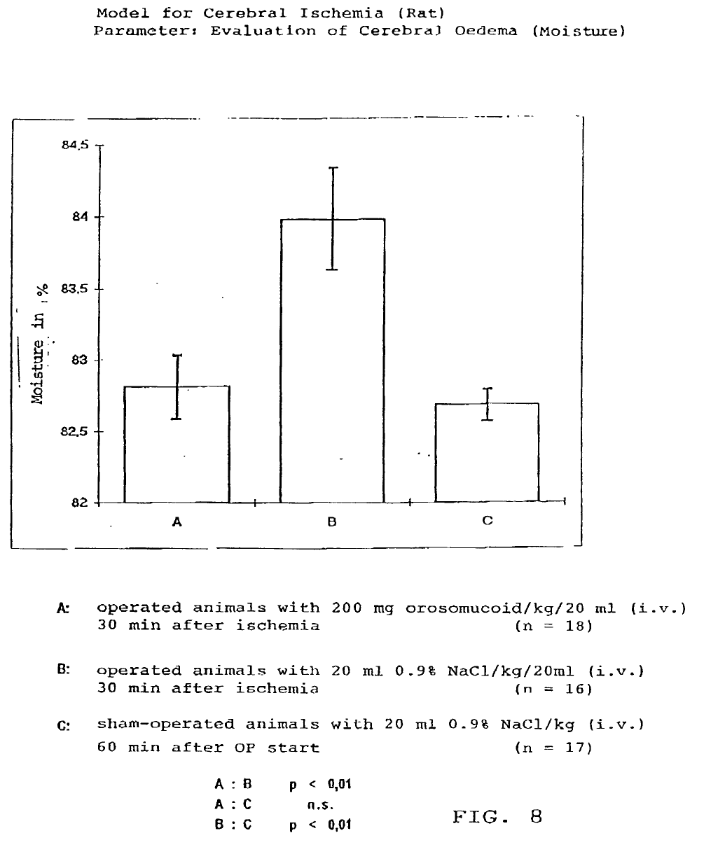

On account of the therapeutic situation in human it has been interesting to check whether orosomucoid, administered after a stroke has occurred, is still effective. Therefore, the dose of 200 mg/kg i.v. found above to be effective was administered 30 minutes after the end of ischemia. As is apparent from FIG. 8 (column A), orosomucoid is fully effective even in this situation.

A protective action of orosomucoid against the cerebral oedema forming as a consequence of a stroke thus has been proven in the animal model.

### EXAMPLE 3

### Treatment of Proteinuria in a Rat Model

Rats were treated i.p. with 100 mg/kg of puromycin aminonucleoside on day 0. Controls received isotonic saline (negative control) in an analogous manner. In metabolic cages, the 24 h urine was collected for protein determination.

The puromycin-treated animals received 200 mg/kg orosomucoid i.v. on the 6th, 7th, 8th and 9th test day, or isotonic saline in analogous manner (positive controls).

On day 10, the animals were weighed and sacrificed by heart puncture for plasma recovery. Kidney wet weight was determined, and creatinine and urea were measured from plasma.

In doing so, the following parameters were found: total protein of urine (mg/24 h), plasma creatinine (mg/dl), blood urea (mg/dl) and the kidney index (kidney weight in % of body weight).

FIG. 9 shows the proteinuria values of the first test run: Animals which had been treated with isotonic saline on day 0 exhibited slight, physiological proteinuria (full diamonds). In animals treated with puromycin, the protein in the urine rose from the third day onwards. In animals treated with isotonic saline on days 6 to 9, the total protein reached 500 to 600 mg/24 h (open triangles) In animals treated with orosomucoid on days 6 to 9, the protein secretion dropped practically to the control values (full squares).

1. The use of human α₁-acid glycoprotein for producing a pharmaceutical preparation for treating non-inflammatory disturbances of circulation or microcirculation, respectively.

2. The use according to claim 1, characterized in that the preparation is suitable for the treatment of hemorrhagic shock and/or hypovolemic shock.

3. The use according to claim 2, characterized in that the preparation is suitable for stabilizing the intravasal volume, in particular in case of acute hemorrhages, excessive loss of liquid or vasodilation, respectively.

4. The use according to claim 1, characterized in that the preparation is suitable for preventing reperfusion damages occurring in connection with a reduced perfusion.

5. The use according to claim 4, characterized in that the preparation is suitable for preventing reperfusion damages as a consequence of a stroke, in particular for reducing cerebral oedema.

6. The use according to claim 1, characterized in that the preparation is suitable for treating microcirculatory disturbances in organs, in particular in the kidney.

7. The use according to claim 6, characterized in that the preparation is suitable for treating proteinuria.

8. The use according to claim 6, characterized in that the preparation is suitable for preventing or treating, respectively, oedemas.

9. The use according to any one of claims 1 to 8, characterized in that the pharmaceutical preparation is produced as a storage-stable infusion solution.

10. The use according to any one of claims 1 to 9, characterized in that the pharmaceutical preparation is produced as a lyophilisate.

11. The use according to any one of claims 1 to 10, characterized in that the pharmaceutical preparation is used in a dose ranging from 70 mg/kg to 5 g/kg, in particular ranging from 100 to 700 mg/kg.

12. The use according to any one of claims 1 to 11, characterized in that the pharmaceutical preparation comprises at least 50% of α₁-acid glycoprotein, in particular more than 70%, preferably more than 90%, based on the total protein.

13. The use according to any one of claims 1 to 12, characterized in that the pharmaceutical preparation further comprises albumin.

14. The use according to any one of claims 1 to 13, characterized in that the pharmaceutical preparation comprises a stabilizer, in particular sodium caprylate.

15. The use according to any one of claims 1 to 14, characterized in that the pharmaceutical preparation is treated for virus inactivation or virus depletion, respectively, in particular by at least one physical treatment, such as heat treatment and/or filtration.

16. The use according to claim 2, characterized in that the treatment comprises an administration of vasoactive substances, in particular catechol amines.

17. The use according to claim 16, characterized in that the vasoactive substances are administered together or in parallel.

18. Infusion preparation for treating shock conditions, comprising α₁-acid glycoprotein and a vasoactive substance as its active components.

19. A kit for treating shock conditions, comprising
a) α₁-acid glycoprotein in a pharmaceutical preparation, and
b) a vasoactive substance.
